(19) United States
(12) Patent Application Publication
(10) Pub. No.: US 2024/0284917 A1
(43) Pub. Date: Aug. 29, 2024

(54) DELIVERY SYSTEMS

(71) Applicant: Firmenich SA, Satigny (CH)

(72) Inventors: Huda JERRI, Plainsboro, NJ (US); Christopher HANSEN, Plainsboro, NJ (US); Jian ZHANG, Plainsboro, NJ (US)

(21) Appl. No.: 18/290,538

(22) PCT Filed: Jun. 28, 2022
(86) PCT No.: PCT/EP22/67744

§ 371 Date: Nov. 14, 2023

Related U.S. Application Data
(60) Provisional application No. 63/216,671, filed on Jun. 30, 2021.

(30) Foreign Application Priority Data
Aug. 12, 2021 (EP) 21191098.9

(51) Int. Cl. A01N 65/08; A01N 25/10; A01N 25/28; A01P 1/00; A61K 8/11; A61K 8/9789; A61Q 5/02

(52) U.S. Cl. CPC A01N 65/08; A01N 25/10; A01N 25/28; A01P 1/00; A61K 8/11; A61K 8/9789; A61Q 5/02

(57) ABSTRACT

Disclosed herein are delivery systems which incorporate Moringa oleifera extract containing Moringa oleifera coagulation protein, therefore providing consumer products which contain natural extracts that provide antimicrobial benefits. Also disclosed herein are delivery systems including a carrier and a hydrophobic formulation, where the carrier includes Moringa oleifera coagulation protein (MOCP). Also disclosed herein are consumer products including the delivery system of the invention.

### INTRODUCTION

The present invention relates to delivery systems. More particularly, the present invention relates to the use an extract from Moringa oleifera containing MOCP as an antimicrobial agent. The delivery system of the invention may be incorporated into consumer products including hygiene and food products.

There is an increasing consumer demand for products which contain natural extracts that provide antimicrobial benefits. Such natural products not only improve the quality of the products in which they are incorporated, but also provide consumer confidence since they are known in nature and hence not perceived as an artificial additive.

Moringa oleifera is a tree of the family Moringaceae and is native to the Indian subcontinent. The tree has been cultivated since antiquity for its beneficial food and health properties. In particular, the Moringa tree provides multifunctional benefits with leaves that are extremely nutritious (Iron, calcium, vitamin C and other micronutrients), a seed which can be pressed to yield cosmetic oil or biofuel, and additionally, a water-soluble cationic protein which has been used as an antimicrobial flocculant for water clarification for centuries. The Moringa tree is a shade-providing, nutritious tree which is vital to communities needing greater access to clean water and nutrition.

The Moringa oleifera plant contains a range of coagulation peptides (Moringa oleifera coagulation proteins (MOCP)) which bind to anionic surfaces and renders certain bacteria and microorganisms unviable. The coagulation proteins can be cationic, and such proteins can be antimicrobial because of the way they electrostatically interact with anionic biosurfaces, membranes and microorganisms. Cationic materials will also likely promote deposition and interaction with biosurfaces and fibers such as skin, hair and natural fabrics which are generally accepted to be negatively-charged, and could be used to boost the deposition performance, substantivity and tenacity of different actives.

In this invention, the present inventors sought to develop delivery systems which incorporate Moringa oleifera extract containing MOCP. In this way the invention provides consumer products which contain natural extracts that provide antimicrobial benefits.

### SUMMARY OF THE INVENTION

In the invention claimed herein, MOCP or an extract from Moringa oleifera comprising MOCP is used as a water soluble emulsifier, and hence functions as a carrier to permit the delivery of hydrophobic formulations, including flavour or fragrance active ingredients.

Therefore a first aspect of the invention provides a delivery system comprising a carrier and a hydrophobic formulation, wherein the carrier comprises Moringa oleifera coagulation protein (MOCP).

Since MOCP is an antimicrobial agent, the delivery system comprises natural antimicrobial activity.

For the avoidance of doubt, the carrier have an outer coating of Moringa oleifera coagulation protein (MOCP).

An embodiment of this aspect of the invention is wherein the MOCP is in an extract from Moringa oleifera.

A further embodiment of the delivery system of the invention is wherein the carrier is a polymeric shell. Preferably the MOCP is on the outside of the polymeric shell, or alternatively the MOCP is formulated within the polymeric shell.

A further embodiment of the delivery system of the invention is wherein the polymeric shell comprising a material selected from aminoplast-based, polyurea-based or polyurethane-based.

A further embodiment of the delivery system of the invention is wherein carrier is a polymeric carrier matrix comprising a material selected from modified starch, maltodextrin, gums, non-animal proteins, polysaccharides and/or soluble fibers.

A further embodiment of the delivery system of the invention is wherein the delivery system is a polymeric carrier matrix comprising the polymeric shell.

A further embodiment of the delivery system of the invention is wherein the delivery system further comprises one or more emulsifying agents and/or antimicrobial agents.

A further embodiment of the delivery system of the invention is wherein the delivery system is in the form of a spray-dried particles.

A further embodiment of the delivery system of the invention is wherein the extract from Moringa oleifera is present at an amount of 1% to 95% by weight of the delivery system.

A further embodiment of the delivery system of the invention is wherein the hydrophobic formulation comprises an active ingredient. Preferably the active ingredient is a perfume, flavor, dye, dye precursor, catalyst from chemical reactions, adhesive, reactive substance for adhesive applications, pharmaceutical active substance, preservative, cosmetic active substance, emollient, conditioner, palliative skin/hair product, plant protection active substance (for example insecticide, fungicide, herbicide, bacteriocide), water repellent, flame retardant, sunscreen agent, solvent or a food ingredient.

A further aspect of the invention provides a consumer product comprising a delivery system of the invention. Preferably the consumer product is perfuming consumer product or a flavored consumer product. Preferably the consumer product is a food product comprising a delivery system of the invention in the form of a dried beverage.

A further aspect of the invention provides a delivery system of the invention for use as an antimicrobial agent.

A further aspect of the invention comprises a method for reducing microbial growth comprising the delivery system of the invention. In some embodiments the method may include incorporating the delivery system of the invention into a hygiene product for consumer use (for example surface cleaning products) or incorporating the delivery system of the invention into a food product.

### DESCRIPTION OF THE FIGURES

FIG. 1. Extruded particles, Left, example N; Right, example O. Scale bar represents 5 mm.

### DESCRIPTION OF THE INVENTION

Unless stated otherwise, percentages (%) are meant to designate percent by weight of a composition.

By the term “antimicrobial agent”, it is meant the normal meaning in the art; i.e. an agent which kills microorganism or reduces their growth. As used herein by “microorganisms” we include bacteria, archaea, fungi, protozoa, algae, and viruses.

When referring to “particles” or “powdered composition”, percentages (%) are given for the dried composition.

By “delivery system” it is herein understood to protect active ingredients, in particular of a perfume formulation and the perfume comprised within the perfume formulation, and/or to control their release.

A “microcapsule”, or the similar, in the present invention it is meant that capsules have a particle size distribution in the micron range (e.g. a mean diameter (d(v, 0.5)) comprised between about 1 and 3000 μm, preferably between 1 and 500 μm, more preferably between 1 and 50 μm) and comprise an external polymeric shell and an internal continuous oil phase enclosed by the external shell. Coacervates are also part of the present invention

The present invention is directed to a delivery system which incorporates a carrier comprising Moringa oleifera coagulation protein and various embodiments and uses of that carrier system.

Moringa oleifera coagulation protein has antimicrobial activity and is natural, vegan, plant-derived and associated with health and wellness, it offers a versatile means to provide antimicrobial benefits to a wide range of flavor and perfumery applications, including confectionary, oral care, beverage, home care, surface care, body/hair care (rinse-offs, deodorant/AP, dry shampoo), fabric/laundry care and air care. Moreover since MOCP is a natural emulsifying agent it can be incorporated into many different delivery technologies, including spray dried powders and microcapsules to bring anti-microbial benefits to many consumer applications.

There are very few known natural water soluble emulsifying agents having antimicrobial activity, and can be combined with other natural antimicrobial oil, for example oils (eucalyptus, peppermint, lemongrass, orange, rosemary, thyme, etc.)

In the invention claimed herein, MOCP or an extract from Moringa oleifera comprising MOCP is used as an emulsifier, and hence functions as a carrier to permit the delivery of hydrophobic formulations, including flavour or fragrance active ingredients.

Moringa oleifera Coagulation Protein

As stated above, the Moringa oleifera plant contain one or more a cationic double helix peptides (the primary function peptides are termed Moringa oleifera coagulation protein (MOCPs)) which bind to anionic surfaces and renders certain bacteria and microorganisms unviable. Hence MOCPs can be used as a natural antimicrobial agent.

MOCPs are well known and have been genetically characterized for a number of years. For example, Samineni et al (2019) Environ. Sci. Technology 53, 12706-12714 and Freire et al (2015) PLOS ONE PLOS ONE|DOI:10.1371/journal.pone.0119871 provide information on a number of MOCP peptides.

Examples of MOCPs which can be used in the present invention include Mo2.1 (Seq ID No: 1), MoCBP (Seq ID No: 2), MoCBP3.1 (Seq ID No. 3), MoCBP3.2 (Seq ID No: 4), MoCBP3.3 (Seq ID No: 5) and MoCBP3.4 (Seq ID No: 6). However all MOCPs from Moringa are encompassed by this aspect of the invention.

Therefore, the Moringa oleifera coagulation protein used in the delivery system of the invention may be prepared using molecular biology methods.

The nucleic acid sequences encoding MOCP proteins can be inserted in expression vectors and/or be contained in chimeric genes inserted in expression vectors, to produce MOCP in a host cell or non-human host organism. The vectors for inserting transgenes into the genome of host cells are well known in the art and include plasmids, viruses, cosmids and artificial chromosomes. Binary or co-integration vectors into which a chimeric gene is inserted can also be used for transforming host cells.

Non-human host organisms suitable to carry out the production of MOCPs vivo may be any non-human multicellular or unicellular organisms. In one embodiment, the non-human host organism used to carry out an embodiment herein in vivo is a plant, a prokaryote or a fungus. Any plant, prokaryote or fungus can be used. In another embodiment the non-human host organism used to carry out the method of an embodiment herein in vivo is a microorganism. Any microorganism can be used, for example, the microorganism can be a bacteria or yeast, such as E. coli or Saccharomyces cerevisiae.

To prepare MOCP proteins the host organism or host cell is cultivated under conditions conducive to the production MOCP proteins. If the host is a unicellular organism, conditions conducive to the production of a MOCP proteins may comprise addition of suitable cofactors to the culture medium of the host. In addition, a culture medium may be selected, so as to maximize MOCP proteins synthesis.

Once produced, MOCP proteins can be isolated from the host cells using standard protein purification methods. For example standard chromatography methods can be used to prepare the MOCP proteins.

Alternatively the MOCP is in an extract from Moringa oleifera

MOCP is present in extracts from, for example, seed coating and leaf tissues. Moringa seed protein and leaf protein powders are available from many sources (for example Lifetime Tea, Chandler, AZ, USA).

Examples of how Moringa oleifera extracts comprising MOCP can be prepared for use in the present invention are provided in the accompanying examples and also below.

A quantity of Moringa seed protein powders is added to distilled water at a ratio of around 1 g protein to 2.33 g of distilled water. The solution is mixed in a homogenizer for 5 mins then heated around 60° C. for 30 min. Following mixing again in a homogenizer for 5 mins the solution is cooled to room temperature and pH adjusted to 6 with NaOH solution. The Moringa seed protein preparation is then centrifuged an the supernatant collected. This is a Moringa oleifera extract as used in the present invention.

A quantity of Moringa leaf protein is added to distilled water at a ratio of around 1 g protein to 5.66 g of distilled water. The solution is mixed in a homogenizer for 5 mins then heated around 60° C. for 30 min. Following mixing again in a homogenizer for 5 mins the solution is cooled to room temperature and pH adjusted to 6 with NaOH solution. The Moringa leaf protein preparation is then centrifuged and the supernatant collected. This is also Moringa oleifera extract as used in the present invention.

As shown in the accompanying examples, the present inventors have determined the amount of MOCP in an extract from Moringa oleifera which is needed to provide an antimicrobial effect on the

An additional aspect of the invention is wherein the substrate is treated with the composition in an amount sufficient to provide an antimicrobial effect.

By the term “effect”, it is meant the inhibition of growth of microorganisms or the killing of microorganisms.

Preferably the extract from Moringa oleifera is present at an amount between 1% to 95% of the total weight of the delivery system, more preferably at an amount of 10% to 85% by weight.

### Hydrophobic Formulation

The present invention is directed to a delivery system which incorporates a hydrophobic formulation.

By “hydrophobic formulation”, it is meant any hydrophobic formulation-single ingredient or a mixture of ingredients-which forms a two-phase dispersion when mixed with water.

In a preferred aspect of the invention, the hydrophobic formulation is defined by a log P above 1, more preferably above 2.

Preferably the hydrophobic formulation comprises one or more hydrophobic active ingredient(s). Preferably the active ingredient is a perfume, flavor, dye, dye precursor, catalyst from chemical reactions, adhesive, reactive substance for adhesive applications, pharmaceutical active substance, preservative, cosmetic active substance, emollient, conditioner, palliative skin/hair product, plant protection active substance (for example insecticide, fungicide, herbicide, bacteriocide), water repellent, flame retardant, sunscreen agent, solvent or a food ingredient.

In a preferred aspect of the invention, the active ingredient is selected from flavours and fragrances. For the purpose of the present invention, the terms “flavour or fragrance” encompass flavour or fragrance ingredients or compositions of current use in the flavour and/or fragrance industry, of both natural and synthetic origin. It includes single compounds and mixtures. Specific examples of such flavour or fragrance ingredients may be found in the current literature, e.g. in Fenaroli's Handbook of flavour ingredients, 1975, CRC Press; Synthetic Food adjuncts, 1947 by M. B. Jacobs, edited by Van Nostrand; or Perfume and Flavor Chemicals by S. Arctander, 1969, Montclair, New Jersey (USA). Many other examples of current flavouring and/or perfuming ingredients may be found in the patent and general literature available. The flavouring or perfuming ingredients may be present in the form of a mixture with solvents, adjuvants, additives and/or other components, generally those of current use in the flavours and fragrance industry.

“Flavouring ingredients” are well known to a person skilled in the art of aromatising as being capable of imparting a flavour or taste to a consumer product, or of modifying the taste and/or flavour of said consumer product, or yet its texture or mouthfeel.

By “perfuming ingredients” it is understood here compounds which are used as active ingredients in perfuming preparations or compositions in order to impart a hedonic effect when applied to a surface. In other words, such compounds, to be considered as being perfuming ones, must be recognized by a person skilled in the art of perfumery as being able to impart or modify in a positive or pleasant way the odor of a composition or of an article or surface, and not just as having an odor. Moreover, this definition is also meant to include compounds that do not necessarily have an odor but are capable of modulating the odor of a perfuming composition, perfumed article or surface and, as a result, of modifying the perception by a user of the odor of such a composition, article or surface. It also contains malodor counteracting ingredients and compositions. By the term “malodor counteracting ingredient” we mean here compounds which are capable of reducing the perception of malodor, i.e. of an odor that is unpleasant or offensive to the human nose by counteracting and/or masking malodors. In a particular embodiment, these compounds have the ability to react with key compounds causing known malodors. The reactions result in reduction of the malodor materials' airborne levels and consequent reduction in the perception of the malodor.

Accordingly, in an embodiment, the hydrophobic active ingredient comprises at least 5 wt. %, preferably at least 10.%, preferably at least 20%, more preferably at least 30% and most preferably at least 40% of chemical compounds having a vapour pressure of at least 0.007 Pa at 25° C., preferably at least 0.1 Pa at 25° C., more preferably at least 1 Pa at 25° C. and most preferably at least 10 Pa at 25° C., all percentages being defined by weight relative to the total weight of the hydrophobic active ingredient. Compounds meeting these criteria are generally regarded as having a volatile character and therefore have an odor or flavour. The method of the present invention therefore allows efficient encapsulation of high amounts of volatile ingredients.

According to a particular embodiment, the hydrophobic active is a mixture of a perfume oil and a neutral carrier oil selected from cosmetically acceptable solvents or emollients such as silicon oils, mineral oils, alkanes, paraffin, triglycerides, fatty acids or gums, or mixture thereof. Examples of such products, but not limited to, are Neobee, Ester gum, Damar gum, isopropyl myristate or paraffins such as Gemseal.

According to a particular embodiment, the hydrophobic active is a mixture of a flavour oil and a neutral carrier oil selected from triglycerides, fatty acids or gums, or mixture thereof. Examples of such products, but not limited to, are Neobee, Ester gum, or Damar gum.

For the purpose of the present invention the vapour pressure is determined by calculation. Accordingly, the method disclosed in “EPI suite”; 2000 U.S. Environmental Protection Agency, is used to determine the value of the vapour pressure of a specific compound or component of the hydrophobic active ingredient.

The amount of hydrophobic active ingredient in the delivery system of the invention is preferably comprised between 1% and 90% by weight, more preferably between 10% and 60% by weight, relative to the total weight of the delivery system.

Also when spray dried it can be in the matrix as well as in or on the microcapsules

### Carrier

By delivery system it is herein understood to protect a hydrophobic formulation, in particular of a hydrophobic formulation comprising an active ingredient, and/or to control their release.

By carrier or carrier material is herein understood that the material of the carrier is suitable to entrap, encapsulate or hold a certain amount of hydrophobic formulation. In order to be qualified as a carrier material, the carrier material has to entrap, encapsulate or hold a certain amount of hydrophobic formulation.

Typically, when the delivery system is in a matrix form, the carrier material is a matrix material and the delivery system has to entrap preferably at least 5 wt. %, preferably at least 10 wt. %, even more preferably at least 15 wt. % of the hydrophobic formulation, based on the total weight of the delivery system.

An embodiment of the present invention is wherein the carrier is a core shell microcapsule.

In such an embodiment the MOCP is on the outside of the core shell microcapsule.

In another embodiment the MOCP is formulated within the core shell microcapsule. By “within” the present invention includes where the MOCP is formulated to be encapsulated, within the capsule, and also where the MOCP is incorporated into the material forming the shell itself.

Typically, when the delivery system is in the form a core-shell microcapsule, the carrier is a shell which comprises between 1% to 50% of the weight of the delivery system.

When the delivery system is in the form a spray dried powder the hydrophobic formulation comprises between 10% to 30% of the weight of the delivery system.

When the microcapsules are in the form of a slurry with water, then the amount of hydrophobic formulation will be less than 50% of the total weight of the delivery system.

In a particular embodiment, the carrier or carrier material is a solid carrier material, i.e. an emulsion or solvent is not a carrier or carrier material.

In a particular embodiment, the delivery system is a core-shell microcapsule or the delivery system is in a matrix form (i.e hydrophobic formulation entrapped within a polymeric matrix, for example a monomeric, oligomeric or polymeric carrier matrix). For the sake of clarity, thereby it is understood that when the delivery system is a core-shell microcapsule, the hydrophobic formulation is comprised in the core which is surrounded or entrapped by the shell. When the delivery system is in the form of a matrix, the hydrophobic formulation is entrapped in a matrix of a carrier, such as a monomeric, oligomeric or polymeric carrier matrix, by adsorption in the matrix.

In case the carrier is a monomeric, oligomeric or polymeric carrier matrix, it is herein understood that the hydrophobic formulation is entrapped in the monomeric, oligomeric or polymeric carrier matrix by dispersion within the monomeric, oligomeric or polymeric carrier matrix.

In a particular embodiment, the carrier material comprises a monomeric, oligomeric or polymeric carrier material, or mixtures of two or more of these. An oligomeric carrier is a carrier wherein 2-10 monomeric units are linked by covalent bonds. For example, if the oligomeric carrier is a carbohydrate, the oligomeric carrier may be sucrose, lactose, raffinose, maltose, trehalose, maltodextrin, and fructo-oligosaccharides.

Examples of a monomeric carrier materials are glucose, fructose, mannose, galactose, arabinose, fucose, sorbitol, mannitol, for example.

Polymeric carriers have more than 10 monomeric units that are linked by covalent bonds.

Non limiting examples of the latter include polyvinyl acetate, polyvinyl alcohol, dextrines, maltodextrines, natural or modified starch, vegetable gums, pectins, xanthanes, alginates, carragenans or yet cellulose derivatives such as for example carboxymethyl cellulose, methylcellulose or hydroxyethylcellulose, and generally all materials currently used for encapsulation of volatile substances. Preferably, the polymeric carrier comprises maltodextrin. According to a particular embodiment, it comprises maltodextrin and modified starch, such as, for example, alkenyl-succinated starch.

The carrier material is preferably present in an amount between 25 and 95 wt. %, preferably between 30 and 60 wt. % and more preferably between 40 and 55 wt. % (based on the total weight of the delivery system).

In a preferred embodiment, the polymeric carrier material may further comprise a fireproofing agent, preferably selected from the group consisting of sodium silicate, potassium silicate, sodium carbonate, sodium hydrogencarbonate, monoammonium phosphate or carbonate, diammonium phosphate, mono-, di- or trisodium phosphate, sodium hypophosphite, melamine cyanurate, chlorinated hydrocarbons, talc and mixtures thereof.

In case the delivery system is a core-shell microcapsule having a shell, it is herein understood that the hydrophobic formulation is comprised in the core which is surrounded by a shell of the microcapsule.

The nature of the polymeric shell from the microcapsules of the invention can vary. As non-limiting examples, the shell can be aminoplast-based, polyurea-based or polyurethane-based. The shell can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an embodiment, the shell comprises an aminoplast copolymer, such as melamine-formaldehyde or urea-formaldehyde or cross-linked melamine formaldehyde or melamine glyoxal.

According to another embodiment the shell is polyurea-based made from, for example but not limited to isocyanate-based monomers and amine-containing crosslinkers such as guanidine carbonate and/or guanazole. Preferred polyurea microcapsules comprise a polyurea wall which is the reaction product of the polymerisation between at least one polyisocyanate comprising at least two isocyanate functional groups and at least one reactant selected from the group consisting of an amine (for example a water soluble guanidine salt and guanidine); a colloidal stabilizer or emulsifier; and an encapsulated perfume. However, the use of an amine can be omitted.

According to a particular embodiment the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another embodiment, the emulsifier is an anionic or amphiphilic biopolymer preferably chosen from the group consisting of gum Arabic, soy protein, gelatin, sodium caseinate and mixtures thereof.

According to another embodiment, the shell is polyurethane-based made from, for example but not limited to polyisocyanate and polyols, polyamide, polyester, etc.

The preparation of an aqueous dispersion/slurry of core-shell microcapsules is well known by a skilled person in the art. In one aspect, said microcapsule wall material may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

According to a particular embodiment, the core-shell microcapsule is a formaldehyde-free capsule. A typical process for the preparation of aminoplast formaldehyde-free microcapsules slurry comprises the steps of
1) preparing an oligomeric composition comprising the reaction product of, or obtainable by reacting together
a) a polyamine component in the form of melamine or of a mixture of melamine and at least one C₁-C₄ compound comprising two NH2 functional groups;
b) an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
c) a protic acid catalyst;
2) preparing an oil-in-water dispersion, wherein the droplet size is comprised between 1 and 600 μm, and comprising:
   I. an oil;
   II. a water medium
   III. at least an oligomeric composition as obtained in step 1;
   IV. at least a cross-linker selected amongst
A. C₄-C₁₂ aromatic or aliphatic di- or tri-isocyanates and their biurets, triurets, trimmers, trimethylol propane-adduct and mixtures thereof; and/or
B. a di- or tri-oxiran compounds of formula

A-(oxiran-2-ylmethyl)ₙ
wherein n stands for 2 or 3 and 1 represents a C₂-C₆ group optionally comprising from 2 to 6 nitrogen and/or oxygen atoms;
   i. optionally a C₁-C₄ compounds comprising two NH2 functional groups;
3) Heating said dispersion;
4) Cooling said dispersion.

This process is described in more details in WO 2013/068255, the content of which is included by reference.

According to another embodiment, the shell of the microcapsule is polyurea- or polyurethane-based. Examples of processes for the preparation of polyurea and polyureathane-based microcapsule slurry are for instance described in WO2007/004166, EP 2300146, EP2579976 the contents of which is also included by reference. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 μm;
d) Applying conditions sufficient to induce interfacial polymerisation and form microcapsules in form of a slurry.

### Preferred Embodiments of the Delivery System

A preferred embodiment of the invention is wherein the delivery system is a matrix carrier format and has the following composition

TABLE 1
Composition of spray dried powders
containing Moringa seed extract

| | Range/ratio/ | Preferred Range/ratio/ |
|---|---|---|
| Feature | Parameter | Parameter |
| Moringa seed extract | 10-95% | 20-85% |
| Hydrophobic formulation | 5-50% | 15-30% |
| Additional emulisifer (for | 0-5% | 0.5-2% |
| example) Q-Naturale | | |
| Others | 0-85% | 0-50% |

Hence a preferred embodiment of the present invention is wherein the delivery system is a matrix carrier and comprises 10% to 95% Moringa seed extract (preferably 20% to 85%), 5% to 50% hydrophobic formulation (preferably 15% to 30%), 0% to 5% additional emulsifier (preferably 0.5% to 2%) and 0% to 85% of other components as listed herein (preferably 0% to 50%).

A further preferred embodiment of the invention is wherein the delivery system is a core shell microcapsule format and has the following composition

TABLE 2
Composition of microcapsules containing Moringa seed extract

| | Range/ratio/ | Preferred Range/ratio/ |
|---|---|---|
| Feature | Parameter | Parameter |
| Moringa seed extract | 1-30% | 10-20% |
| Hydrophobic formulation | 10-60% | 20-50% |
| Polyisocyanate monomer | 0.1-9% | 0.2-7.5% |
| Water | 50-85% | 55-70% |
| Additional emulsifier | 0-5% | 0-2% |

Hence a preferred embodiment of the present invention is wherein the core shell microcapsule format and comprises 1% to 30% Moringa seed extract (preferably 10% to 20%), 10% to 60% hydrophobic formulation (preferably 20% to 50%), 0.1% to 9% polyisocyanate monomer (preferably 0.2% to 7.5%), 0% to 5% water (preferably 0% to 2%) water and 0% to 5% additional emulsifier (preferably 0% to 2%)

### Further Components of the Delivery System of the Invention

An embodiment of the invention is wherein the delivery system further comprises one or more emulsifying agents and/or antimicrobial agents,

By “emulsifying agents” we include modified starch, gums, proteins, saponins and similar such agents as well known in the art.

By antimicrobial agents” we include alkaloids, phenolics, essential oil, saponins, chitosan, nisin, lauric arginate and similar such agents as well known in the art.

### Consumer Products

A further aspect of the invention provides a consumer product comprising a delivery system the invention.

Preferably the consumer product is perfuming consumer product or a flavored consumer product.

Non-limiting examples of suitable perfuming consumer product can be a perfume, such as a fine perfume, a splash or eau de perfume, a cologne or a shave or after-shave lotion; a fabric care product, such as a liquid or solid detergent, a fabric softener, a fabric refresher, an ironing water, a paper, or a bleach, carpet cleaners, curtain-care products; a body-care product, such as a hair care product (e.g. a shampoo, a coloring preparation or a hair spray, a color care product, hair shaping product, a dental care product), a disinfectant, an intimate care product; a cosmetic preparation (e.g. a skin cream or lotion, a vanishing cream or a deodorant or antiperspirant (e.g. a spray or roll on), hair remover, tanning or sun or after sun product, nail products, skin cleansing, a makeup); or a skin-care product (e.g. a perfumed soap, shower or bath mousse, oil or gel, or a hygiene product or a foot/hand care products and hand sanitizers); an air care product, such as an air freshener or a “ready to use” powdered air freshener which can be used in the home space (rooms, refrigerators, cupboards, shoes or car) and/or in a public space (halls, hotels, malls, etc. . . . ); or a home care product, such as a mold remover, furnisher care, wipe, surface coating for face masks, a dish detergent or hard-surface (e.g. a floor, bath, sanitary or a windows) detergent; a leather care product; a car care product, such as a polish, waxes or a plastic cleaners. Alternatively, in some aspects, the consumer products are body care or home care products.

Furthermore, the delivery system of the invention can be added to a flavored consumer product

For the sake of clarity, by “flavored consumer product” it is meant to designate an edible product which may be food or beverage and which can be fried or not, as well as frozen or not, low fat or not, marinated, battered, chilled, dehydrated, instant, canned, reconstituted, retorted or preserved. Therefore, a flavored article according to the invention comprises the invention's extract, as well as optional benefit agents, corresponding to taste and flavor profile of the desired edible product, e.g. a savory cube.

The nature and type of the constituents of the foodstuffs or beverages do not warrant a more detailed description here, the skilled person being able to select them on the basis of his general knowledge and according to the nature of said product.

Typical examples of said flavored consumer product include:
seasoning or condiment, such as a stock, a savory cube, a powder mix, a flavored oil, a sauce (e.g. a relish, a barbecue sauce, a dressing, a gravy or a sweet and/or a sour sauce), a salad dressing or a mayonnaise;
meat-based product, such as a poultry, beef or pork based product, a seafood, surimi, or a fish sausage;
soup, such as a clear soup, a cream soup, a chicken or beef soup or a tomato or asparagus soup;
carbohydrate-based product, such as instant noodles, rice, pasta, potatoes flakes or fried, noodles, pizza, tortillas, wraps;
dairy or fat product, such as a spread, a cheese, or regular or low fat margarine, a butter/margarine blend, a butter, a peanut butter, a shortening, a processed or flavored cheese;
savory product, such as a snack, a biscuit (e.g. chips or crisps) or an egg product, a potato/tortilla chip, a microwave popcorn, nuts, a bretzel, a rice cake, a rice cracker, etc;
imitation products, such as a dairy (e.g a reformed cheese made from oils, fats and thickeners) or seafood or meat (e.g. a vegetarian meat replacer, a veggie burger) or analogues;
pet or animal food; or
beverage such as a hot drink (e.g. a tea or coffee), a soft drink including carbonated, an alcoholic drink (e.g. whisky), a ready-to-drink or a powder soft.

In a preferred embodiment, the delivery system of the invention is added to a chewing gum or other similar product.

In a further preferred embodiment the delivery system of the invention is prepared in a dry flavored consumer product, for example a dried beverage.

The proportions in which the delivery system of the invention can be incorporated into the various of the aforementioned products vary within a wide range of values. These values are dependent on the nature of the consumer product to be flavored and on the desired organoleptic effect as well as the nature of the co-ingredients in a given base when the composition according to the invention are mixed with perfuming or flavoring ingredients, solvents or additives commonly used in the art.

For example, in the case of flavored consumer product, typical concentrations are in the order of 10 ppm to 100,000 ppm, more preferably 1000 ppm to 50,000 ppm, even more preferably 3000 ppm to 10000 ppm of the delivery system of the invention based on the weight of the consumer product into which they are incorporated.

The invention will now be described in further detail by way of the following examples which illustrate the benefits and advantages of the present invention.

### EXAMPLES

The invention will now be described in further detail by way of the following examples which illustrate the benefits and advantages of the present invention.

Method. Protein Content & Protein Solubility

Moringa seed protein and leaf protein powders were obtained from Lifetime Tea (Chandler, AZ, USA). Compositional analysis including percent carbon, hydrogen, and nitrogen were conducted for both Moringa seed and leaf proteins. The total protein content was calculated using a multiplication factor of 6.25 on the nitrogen content. Protein solubility of Moringa seed protein was determined by making up 200 grams of aqueous solutions containing 20% wt. of protein powder with D.I. water. Triplicate solution samples were prepared and thoroughly mixed at 60° C. for 5 min to ensure complete hydration. The hydrated solution was adjust to pH 6.0 with 3.0% sodium hydroxide. Then the protein solutions were centrifuged at 6000 RPM for 5 minutes to separate soluble and insoluble fractions. All supernatant was removed and the exact weight was recorded. Compositional analysis was conducted for these supernatants to determine the nitrogen content. The resulting protein content from the solution samples were used with the protein content determined for the powder samples (i.e. 52% for Moringa seed protein powder) to calculate the amount of soluble protein and protein solubility. The results are shown in below table. Moringa seed protein powder has much higher protein content than Moringa leaf protein (52% vs. 24% wt.). The 20% Moringa seed protein solution has protein solubility of 66% wt.

TABLE 3
Protein content and solubility of Moringa seed and leaf protein

| | | Protein solubility |
|---|---|---|
| | Protein content | (20% solution) |
| Moringa seed protein | 52% | 66% |
| Moringa leaf protein | 24% | N/A |

### Example 1: Preparation of Soluble Moringa Seed Protein

Moringa seed protein was obtained from Lifetime Tea (Chandler, AZ, USA). The protein content is 52% by weight determined by nitrogen measurement with a factor of 6.25 (assuming proteins have nitrogen content of 16%). Soluble Moringa seed protein was prepared following the below protocol.
1. Add 60 grams of Moringa seed protein to 140 grams of D.I. water in a 500 mL container.
2. Mix the solution using a Silverson L4RT homogenizer at 7000 rpm for 5 min
3. Hold the protein solution in water batch at 60° C. for 30 min
4. Re-mixing the solution using at 7000 rpm for 5 min
5. Cooling down to room temperature and adjust the pH to 6 with 3% NaOH solution
6. Centrifuge the samples at 6000 rpm for 5 min
7. The supernatant is collected
8. Part of the collected supernatant is freeze dried to obtain soluble Moringa protein (A-1)
9. Part of the collected supernatant is spray dried to obtain soluble Moringa protein (A-2). A mini Buchi dryer B-290 was used with inlet air temperature of about 180° C. and outlet temperature of about 85° C.

### Example 2: Preparation of Soluble Moringa Leaf Protein

Moringa leaf protein was obtained from Lifetime Tea (Chandler, AZ, USA). The protein content is 24% by weight determined by nitrogen measurement with a factor of 6.25 (assuming proteins have nitrogen content of 16%). Soluble Moringa leaf protein was prepared following the below protocol.
1. Add 30 grams of Moringa seed protein to 170 grams of D.I. water in a 500 mL container.
2. Mix the solution using a Silverson L4RT homogenizer at 7000 rpm for 5 min
3. Hold the protein solution in water batch at 60° C. for 30 min
4. Re-mixing the solution using at 7000 rpm for 5 min
5. Cooling down to room temperature and adjust the pH to 6 with 3% NaOH solution
6. Centrifuge the samples at 6000 rpm for 5 min
7. The supernatant is collected and freeze dried to obtain soluble Moringa leaf protein (B)

### Example 3: Preparation of Spray Dried Medium Chain Triglyceride (MCT) Powders

MCT emulsions were prepared by mixing and homogenizing (Silverson L4TR at 7000 rpm for 5 min) MCT with various wall material solutions. Emulsion compositions are described in below table. Spray dried MCT powders were prepared by spray drying prepared MCT emulsions with a mini Buchi dryer B-290 at inlet air temperature of about 180° C. and outlet air temperature of about 85° C.

TABLE 4
Composition of spray dried MCT with various carrier materials

| Ingredient | Example C | Example D | | Example E | Example F |
|---|---|---|---|---|---|
| MCT ¹⁾ | 10 g | 10 | g | 10 g | 5 g |
| Eugenol ²⁾ | | | | | 5 g |
| Maltodextrin ³⁾ | | | | 40 g | |
| Soluble Moringa | 40 g | 37.3 | g | | 40 |
| seed protein ⁴⁾ | | | | | |
| Quillaja | | 2.7 | g | | |
| saponins ⁵⁾ | | | | | |
| D.I water | 150 g | 150 | g | 150 g | 150 g |

¹⁾ Neobee ®M-5; origin: Stephan, USA
²⁾ Eugenol, Firmenich
³⁾ Maltodextrin 10 DE; origin: Cargill, USA
⁴⁾ Prepared from example A-1
⁵⁾ Q-Naturale ® 200; origin: Ingredion, USA

### Example 4: Preparation of Moringa Microcapsules

Protocol 1 Preparation of Microcapsules Functionalized with Moringa

Microcapsules were prepared following the process below and then post functionalized by addition of 20 wt % soluble Moringa seed powder (as previously described).
1) Dissolved gum Arabic in DI water
2) Dissolved Takenate D-110N (Trimethylol propane-adduct of xylylene diisocyanate, origin: Mitsui Chemicals, Inc., Japan, 75% solution of polyisocyanate in ethyl acetate) in Dorisyl containing 5 wt % Uvinul A Plus (UV tracer used for deposition)
3) Added oil phase to aqueous phase and homogenized for 2 min at 10,000 rpm (Ultra-Turrax T25, 18G probe)
4) Heated emulsion for 4 hr at 70° C. and then cooled to RT

### Protocol 2 Preparation of Moringa Microcapsules

Microcapsules were prepared following the process below.
1) Dissolved soluble Moringa seed powder (Lifetime Tea, process previously described) in DI water
2) Dissolved Takenate D-110N in Dorisyl containing 5 wt % Uvinul A Plus (UV tracer used for deposition)
3) Added oil phase to aqueous phase and homogenized for 2 min at 10,000 rpm (Ultra-Turrax T25, 18G probe)
4) Heated emulsion for 4 hr at 70° C. and then cooled to RT

The formulations of the microcapsules can be found in the table below:

TABLE 5
Composition of prepared microcapsules

| Examples | G | H | I | J |
|---|---|---|---|---|
| Sample description | Microcapsule | Microcapsule + | Microcapsule + | Microcapsule + |
| | (control) | 5% Moringa | 10% Moringa | Moringa |
| | | Extract | Extract | |
| Process | Protocol 1 | Protocol 1 | Protocol 1 | Protocol 2 |
| gum Arabic ¹⁾ | 1.0 | 0.75 | 0.5 | — |
| Soluble Moringa seed | — | 5.0 | 10.0 | 16.0 |
| protein ²⁾ | | | | |
| DI Water | 67.5 | 70.61 | 73.75 | 63.0 |
| Dorisyl ³⁾ | 28.5 | 21.38 | 14.25 | 19.0 |
| Uvinul ⁴⁾ | 1.5 | 1.13 | 0.75 | 1.0 |
| Takenate ⁵⁾ | 1.5 | 1.13 | 0.75 | 1.0 |

¹⁾ Superstab AA; origin: Nexira, USA
²⁾ Prepared from example A-1
³⁾ Dorisyl, Firmenich
⁴⁾ Uvinul A Plus, Firmenich
⁵⁾ Takenate D-110N; origin, Mitsui, JP

### Example 5: Deposition of Microcapsules

### Deposition of Microcapsules Protocol

A 500 mg mini brown Caucasian hair swatch was wet with 40 ml of tap water (39° C.) aimed at the mount with a 140 ml syringe. The excess water was gently squeezed out once and 0.1 mL of a model surfactant mixture (8.6 g of SLES (sodium lauryl ether sulfate), 5.0 g CAPB (cocamidopropyl betaine), 6.3 g of 4 wt % Salcare® SC 60, and 30.1 g DI water, pH 5.5), containing microcapsules loaded with a UV tracer (Uvinul A Plus), was applied with a 100 μL positive displacement pipet. The surfactant mixture was distributed with 10 horizontal and 10 vertical passes using the thumb and pointer fingers of gloved hands. The swatch was then rinsed with 100 ml of tap water (39° C.) with 50 mL applied to each side of the swatch aimed at the mount. The water streams through and down the length of the swatch, sufficiently rinsing and flushing the 10 cm swatch. The excess water was gently squeezed out and the hair swatch was then cut into a pre-weighed 20 mL scintillation vial. This process was repeated in triplicate and then the vials containing the cut hair were dried in a vacuum oven at 50-60° C. (100 Torr) for at least 5 hours. After the drying process, the vials were again weighed to determine the mass of the hair in the vials. Controls were also prepared by adding 0.1 mL of a model surfactant mixture containing microcapsules to an empty vial. 4 mL of 200 proof ethanol were then added to each vial and they were subjected to 60 min of sonication. After sonication, the samples were filtered through a 0.45 μm PTFE filter and analyzed with a HPLC using a UV detector. To determine the percentage of deposition of microcapsules from a model surfactant mixture, the amount of Uvinul extracted from the hair samples was compared to the amount of Uvinul extracted from the control samples. Deposition results are normalized to 400 mg hair and reported as the average of triplicate measurements.

As shown in Table 6, deposition of the both Moringa microcapsules (I and J) was better than the control (G).

TABLE 6
Deposition of Microcapsules on hair

| Example | % Deposition |
|---|---|
| G | 3.6 |
| I | 4.3 |
| J | 8.6 |

### Example 6: Preparation of Spray Dried Moringa Microcapsules

Spray dried Moringa microcapsules was prepared with soluble Moringa seed protein on a mini Buchi dryer B-290 at inlet air temperature of about 180° C. and outlet air temperature of about 85° C. The spray dry formulation was described in below table.

TABLE 7
Composition of microcapsule feed solution for spray dry

| Ingredient | Example K |
|---|---|
| Moringa microcapsules ¹⁾ | 25 g |
| Soluble Moringa seed protein ²⁾ | 15 g |
| D.I water | 60 g |

¹⁾ P(Moringa), prepared in example J
²⁾ Prepared from example A-1

### Example 7: Antimicrobial Evaluation

### Preparation of Bacterial Suspensions

Bacterial suspensions of Escherichia coli ATCC 25922 and Staphylococcus aureus ATCC 6538 were prepared as follows. Stock cultures stored at −80° C. were sub-cultured onto agar plate media, and incubated at 37° C. for 24 h to obtain single colonies. Single colonies of the primary cultures were inoculated onto agar plate media to get secondary cultures. Single colonies of the secondary cultures were inoculated into Mueller Hinton (MH) broth media and incubated at 37° C., 180 rpm overnight. Aliquots of overnight cultures were inoculated into 50 ml of fresh broth media, and incubated at 37° C., 180 rpm. When the OD_{600 nm} reached the target value for each strain (see Table 8), cells were harvested by centrifugation at 5000 rpm for 10 min, and then resuspended in the same fresh broth media at the same volume before the centrifugation. Aliquots (1.1 ml) of each cell suspension were diluted in 200 ml of 2× MH broth media as the bacterial solutions for the MIC test.

TABLE 8
Media, aliquots of overnight culture, and target OD of broth cultures for
the preparation of bacterial suspensions of E. coli and S. aureus

| | | | Aliquots of | | |
|---|---|---|---|---|---|
| | Agar plate | Broth | Overnight | | Target |
| Strains | media | Media | Culture | | OD |
| Escherichia coli | TSA^{a} | MH^{b} | 0.5 | ml | 0.3 |
| ATCC 25922 | | | | | |
| Staphylococcus | TSA | MH | 1 | ml | 0.4 |
| aureus | | | | | |
| ATCC 6538 | | | | | |

Note:
^{a}TSA, Tryptic Soy Agar (BD Cat No. 236950),
^{b}MH, Mueller Hinton Broth (BD Cat No. 211443)

### Preparation of Sample Solutions

Sample solutions of test materials were prepared in MilliQ water for MIC test for E. coli and S. aureus strains. In brief, stock solutions of 10% in MilliQ water, and then 1:2 serial dilutions were prepared in MiliQ water to obtain a total of 6 solutions of the test material. Aliquot (100 μl) of each solution was used for MIC test. The tested final concentrations of each material were 5%, 2.5%, 1.25%, 0.625% and 0.3125%, 0.15625% and 0.0781%.

### MIC and MBC Test Procedures

MIC test were performed in 96 well plates. Aliquots (100 μl) of sample solutions were mixed with 100 μl of bacterial solutions in growth media, at concentrations of 105 to 106 cfu/ml, in wells of the 96 well plates. Three replicates for each solution.

The 96 wells plates were incubated at 37° C., 180 rpm for 24 h. After incubation, 10 μL of 0.2% resazurin were added into each well. The plates were further incubated for 4 h at 37° C. Wells with color changed to pink were regarded as an indication of microbial growth. Minimal inhibitory concentration (MIC) was determined as the lowest concentration where no growth was observed. Average MIC value of the three replicates was calculated against each strain.

Viable cells in each well were enumerated by spiral plate method. In brief, a 10-2 dilution of cell suspension in each well was prepared by transfer aliquots (50 μL) from each well to 4950 μL 0.85% saline solution. Aliquots (50 μL) was plated out onto TSA plate using Eddy Jet 2 spiral plater, E-50 Mode. Minimal bactericidal concentration (MBC) was determined as the lowest concentration that yielded no bacterial colony on a TSA.

### MIC and MBC Test Results

Tested concentrations: 5%, 2.5%, 1.25%, 0.625% and 0.3125%, 0.15625% and 0.0781%

TABLE 9

| | |
|---|---|
| Minimal inhibitory | Minimum bactericidal |
| concentration | concentration |
| (MIC) | (MBC) |

| | E. coli | S. aureus | E. coli | S. aureus |
|---|---|---|---|---|
| | ATCC | ATCC | ATCC | ATCC |
| Test material | 25922 | 6538 | 25922 | 6538 |
| Example A1 | 5.0% | 0.3125% | 5.0% | 0.625% |
| Example A2 | >5.0% | 0.3125% | >5.0% | 0.625% |
| Example B | >5.0% | >5.0% | >5.0% | >5.0% |
| Example C | 5.0% | 0.3125% | 5.0% | 0.625% |
| Example D | 2.5% | 0.625% | 5.0% | 1.25% |
| Example E | >5.0% | >5.0% | >5.0% | >5.0% |
| Example F | 2.5% | 0.52% | 5.0% | 5.0% |
| Example J | >5.0% | 5.0% | >5.0% | >5.0% |
| Example K | >5.0% | 0.3125% | >5.0% | 2.5% |

Spray dried powder containing maltodextrin and MCT (Example E) did not shown much activity against either E. coli or S. aureus because both MIC and MBC were greater than 5%. This suggests that MCT and maltodextrin are ineffective to E. coli or S. aureus.

The effective concentration of soluble Moringa seed proteins against S. aureus ATCC6538 was determined to be 0.3%-0.6% (Example A1 and A2) whereas soluble Moringa leaf protein (Example B) seems ineffective for E. coli and S. aureus. Examples C contains about 80% Moringa seed proteins and their effective concentration against S. aureus were determined to be 0.3-1.25%. These results clearly demonstrate that soluble Moringa seed proteins incorporated in spray dry delivery system can provide antimicrobial benefits.

Antimicrobial activity of quillaja saponin has been reported in the literature. Example D contains both Moringa seed protein and quillaja saponins and it showed lower MIC against E. coli and higher MIC against S. aureus in comparison with Example C. This suggests incorporation of water soluble antimicrobials with Moringa seed protein may lead to enhanced activity against E. coli

Antimicrobial activity of eugenol has been reported in the literature. Example F contains eugenol in the oil phase and it showed lower MIC against E. coli and higher MIC against S. aureus in comparison with Example C. This suggests combination of Moringa seed protein and hydrophobic antimicrobials can lead to enhanced activity against E. coli.

Microcapsules made with soluble Moringa seed protein showed positive activity against S. aureus. Example J showed high MIC of 5% against S. aureus because of its low concentration of Moringa seed protein (i.e. 16% by weight of the microcapsule slurry). Microcapsule slurries were spray dried with soluble Moringa seed protein to obtain powdered microcapsules which contains about 80% soluble Moringa seed protein. Example K showed MIC of 0.3125% and MBC of 2.5% against S. aureus.

This clearly demonstrates Moringa seed protein can be incorporated into microcapsule to provide antimicrobial properties.

### Example 8: Further Data Supporting the Invention

### Preparation of Moringa Seed Protein Extract

Soluble Moringa seed protein powder was prepared using protocol from example 1 without pH adjustment of the protein solution during extraction process.

### Preparation of Spray Dried Powders

MCT emulsions were prepared by mixing and homogenizing (Silverson L4TR at 7000 rpm for 5 min) MCT with various wall material solutions. Emulsion compositions are described in below Table 10 Spray dried powders were prepared by spray drying prepared MCT emulsions with a mini Buchi dryer B-290 at inlet air temperature of about 180° C. and outlet air temperature of about 85° C. The spray dried powders were used for anti-microbial testing.

TABLE 10
Composition of spray dried MCT with various carrier materials

| Ingredient | Example L | ExampleM | |
|---|---|---|---|
| MCT ¹⁾ | 10 g | 5 | g |
| Eugenol ²⁾ | | 5 | g |
| Soluble Moringa | 40 g | 40 | g |
| seed protein ³⁾ | | | |
| D.I water | 150 g | 150 | g |

Neobee@M-5; origin: Stephan, USA
Eugenol, Firmenich
Freeze dried soluble moringa seed protein extract

### MIC & MBC Test Results of Moringa Samples

### Determination of MIC and MBC for Bacteria Strains Using Broth Dilution Assay

Bacterial suspensions of Escherichia coli, Staphylococcus aureus, Staphylococcus hominis, Corynebacterium striatum were prepared as follows. Stock cultures stored at −80° C. were sub-cultured onto agar plate media, and incubated at 37° C. for 24 h to obtain single colonies. Single colonies of the primary cultures were inoculated onto agar plate media to get secondary cultures. Single colonies of the secondary cultures were inoculated into broth media and incubated at 37° C., 180 rpm overnight. Aliquots of overnight cultures were inoculated into 50 ml of fresh broth media, and incubated at 37° C., 180 rpm. When the OD_{600 nm} reached the target value for each strain (see Table 11), cells were harvested by centrifugation at 5000 rpm for 10 min, and then resuspended in 2× fresh broth media at the same volume before the centrifugation. Aliquots (1.1 ml) of each cell suspension were diluted in 200 ml of 2× broth media as the bacterial solutions for the MIC test and MBC test.

Concentrations of 5%, 2.5%, 1.25%, 0.625% and 0.3125%, 0.15625% and 0.0781% of two samples were tested to determine the MIC and MBC for bacterial strains as described previously. Samples are shown in Table 11

TABLE 11
Media, aliquots of overnight culture, and target OD of broth cultures for
the preparation of bacterial suspensions of E. coli and S. aureus

| | | | Aliquots of | | |
|---|---|---|---|---|---|
| | Agar plate | Broth | Overnight | | Target |
| Strains | media | Media | Culture | | OD |
| E. coli | TSA^{a} | MH^{b} | 0.5 | ml | 0.3 |
| ATCC 25922 | | | | | |
| S. aureus | TSA | MH | 1 | ml | 0.4 |
| ATCC 6538 | | | | | |
| S. homonis | TSA | MH | 1 | ml | 1.3 |
| DSM 20328 | | | | | |
| C. striatum | TSA + 0.5% | BHI + 0.5% | 1 | ml | 3.5 |
| DSM 20668 | Tween80 | Tween80 | | | |

Note:
^{a}TSA, Tryptic Soy Agar,
^{b}MH, Mueller Hinton Broth;
BHI: Brain Heart Infusion broth

### MIC & MBC Results

TABLE 12
MIC of Moringa samples for bacteria

| | Minimal inhibitory concentration (MIC) | | | |
|---|---|---|---|---|
| | E. coli | S. aureus | S. homonis | C. striatum |
| Test Materials | ATCC25922 | ATCC6538 | DSM 20328 | DSM 20668 |
| Example L | >5% | 0.41% | 0.31% | 0.63% |
| Example M | 5% | 0.52% | 0.31% | 0.63% |

TABLE 13
MBC of Moringa samples for bacterial strains

| | Minimal bactericidal concentration (MBC) | | | |
|---|---|---|---|---|
| | E. coli | S. aureus | S. homonis | C. striatum |
| Test Materials | ATCC25922 | ATCC6538 | DSM 20328 | DSM 20668 |
| Example L | >5% | 1.25% | 1.25% | 0.63% |
| Example M | >5% | 2.50% | 2.50% | 0.63% |

E. coli and S. aureus were selected for potential hygiene applications; S. homonis and C. striatum were selected for deodorant (DEO) applications The results show that examples L and M are active against S. aureus, S. homonis, and C. striatum with MIC ranging from 0.31% to 0.63% and MBC ranging from 0.63% to 2.5%. All these results suggest spray dried powders made with Moringa seed protein extract as emulsifier and carrier can deliver antimicrobial properties for multiple purposes, including hygiene applications, oral care, food and drink applications, for APDO for reducing personal malodors, and many further uses.

### Example 9: Extruded Prototypes

A ZSE 18 co-rotating twin-screw extruder (L/D 18, Leistritz, Branchburg, NJ, USA) was used to encapsulate medium chain triglycerides (MCT) using Moringa seed protein as carrier material. The extruder is equipped with 8 barrels and their temperatures are independently controlled.

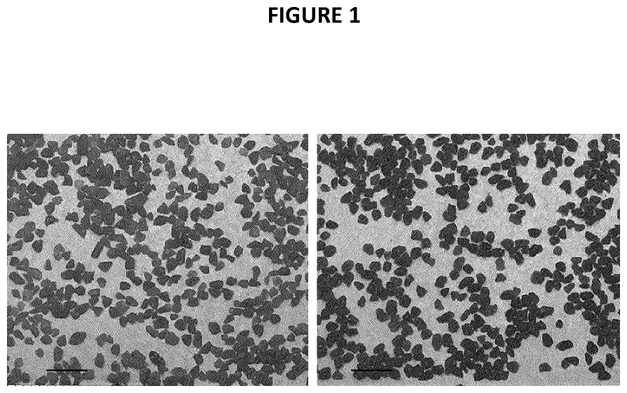

All materials were pre-mixed uniformly in a Stephan mixer. This mixture was then fed into the extruder by means of a loss-in-weight feeder with a flow rate of 2.5 kg/hr. A small amount of water was injected into the extruder in order to obtain glass transition temperature (T_{g}) above 20° C. for the extruded particles. Temperature set points of the extruder barrels from feed to die end ranged from 20 to 100° C. The screw speed was kept constant at 200 rpm. The melt was extruded through a die plate with 2.5 mm diameter holes. After establishing steady-state extrusion condition, strands exiting the die were collected and grinded to different particle size. The samples were kept at room temperature (about 23° C.) and no caking was observed after 2 weeks of storage as shown in FIG. 1.

TABLE 14
Twin screw extrusion formulation.

| | Examples | | |
|---|---|---|---|
| Composition | | N | O |

| | | | | |
|---|---|---|---|---|
| MCT^{a} | 0.12 | kg | 0.08 | kg |
| Moringa seed protein^{b} | 1.38 | kg | 1.84 | kg |
| Maltodextrin 10 DE^{c} | 1.38 | kg | | |
| Water | 0.06 | kg | 0.04 | kg |
| Lubricant | 0.06 | kg | 0.04 | kg |
| Total | 3.0 | kg | 2.0 | kg |

^{a}Medium chain triglyceride (MCT) was from Firmenich, NJ, USA
^{b}Moringa seed protein, Lifetime Tea, AZ, USA
^{c}Maltodextrin with dextrose equivalent (DE) value of 10 (known as 10 DE) was purchased from Cargill, MN, USA

Protein sequences used in the invention
MO2.1
(Seq ID No: 1)
QGPGRQPDFQRCGQQLRNISPPQRCPSLRQAVQLTHQQQGQVGPQQVRQ
MYRVASNIPST
MoCBP
(Seq ID No: 2)
MAKITLLLATFGLLLLLTNASIYRTTVELDEEADENQQQRCRQQFQTHQ
RLRACQRFIRRRTQGGGPLDEVEDEVDEIEEVVEPDQGPGRQPAFQRCC
QQLRNISPPCRCPSLRQAVQLTHQQQGQVGPQQVRQMYRVASNIPSMCN
LQPMSCLFRQQQSSWL
MoCBP3.1
(Seq ID No: 3)
MAKLTLLLATFALLVLLANASIYRTTVELDEEPDDNQQQRCRHQFQSQQ
RLRACQRVIRRWSQGGGPMEDVEDEIGETDEIEEVVEPDQARRPPTLQR
CCRQLRNVSPFCRCPSLRQAVQSAQQQQGQVGPQQVGHMYRVASRIPAI
CNPQPMRCPFRQQQGS
MoCBP3.2
(Seq ID No: 4)
MAKITLLLATFGLLLLLTNASIYRTTVELDEEADENQQQRCRQQFQTHQ
RLRACQRFIRRRTQGGGPLDEVEDEVDEIEEVVEPDQGPGRQPAFQRCC
QQLRNISPPCRCPSLRQAVQLTHQQQGQVGPQQVRQMYRVASNIPSMCN
LQPMSCLFRQQQSSWL
MoCBP3.3
(Seq ID No: 5)
MAKFTLLLAIFALFLILANANVYRTTVELDEEPDDNQQGQQQQQCRQQF
LTHQRLRACQRFIRRQTQGGGALEDVEDDVEEIEEVVEPDQARRPAIQR
CCQQLRNIQPRCRCPSLRQAVQLAHQQQGQVGPQQVRQMYRLASNIPAI
CNLRPMSCPFGQQ
MoCBP3.4
(Seq ID No: 6)
MAKLTLLLATLALLVLLANASIYRTTVELDEEPDDNQQQRCRHQFQTQQ
RLRACQRVIRRWSQGGGPMEDVEDEIDETDEIEEVVEPDQARRPPTLQR
CCRQLRNVSPFCRCPSLRQAVQSAQQQQGQVGPQQVGHMYRVASRIPAI
CNLQPMRCPFRQQQSS

1. A delivery system comprising a carrier and a hydrophobic formulation, wherein the carrier comprises Moringa oleifera coagulation protein (MOCP).

2. The delivery system of claim 1, wherein the MOCP is in an extract from Moringa oleifera.

3. The delivery system according to claim 1, wherein the carrier is a core shell microcapsule.

4. The delivery system according to claim 3, wherein the MOCP is on an outside of the core shell microcapsule.

5. The delivery system according to claim 2, wherein MOCP is formulated within the core shell microcapsule.

6. The delivery system of claim 3 wherein the core shell microcapsule comprises a material that is aminoplast-based, polyurea-based or polyurethane-based.

7. The delivery system of claim 1, wherein the carrier is a polymeric carrier matrix comprising a material selected from the group consisting of modified starch, maltodextrin, gums, proteins, polysaccharides and soluble fibers.

8. The delivery system of claim 1, wherein the delivery system is a polymeric carrier matrix comprising the polymeric shell.

9. The delivery system of claim 1, wherein the delivery system further comprises one or more emulsifying agents and/or antimicrobial agents.

10. The delivery system according to claim 1, wherein the delivery system is in the form of a spray-dried particle.

11. The delivery system of claim 1, wherein the extract from Moringa oleifera is present at an amount of 1% to 95% of the total weight of the delivery system.

12. The delivery system of claim 1, wherein the hydrophobic formulation comprises one or more active ingredient(s).

13. The delivery system of claim 12, wherein the active ingredient(s) is a perfume, flavor, dye, dye precursor, catalyst from chemical reactions, adhesive, reactive substance for adhesive applications, pharmaceutical active substance, preservative, cosmetic active substance, emollient, conditioner, palliative skin/hair product, plant protection active substance (for example insecticide, fungicide, herbicide, bacteriocide), water repellent, flame retardant, sunscreen agent, solvent or a food ingredient.

14. A consumer product comprising the delivery system of claim 1.

15. The consumer product of claim 12, wherein said product is a perfuming consumer product or a flavored consumer product.

17. An antimicrobial agent comprising the delivery system of claim 1.

18. A method for reducing microbial growth, comprising incorporating the delivery system of claim 1 into a hygiene product for consumer use or into a food product.


SEQUENCE LISTING (6 sequences)

SEQ ID NO: 1 - protein, 60 aa, Moringa oleifera
QGPGRQPDFQRCGQQLRNISPPQRCPSLRQAVQLTHQQQGQVGPQQVRQMYRVASNIPST

SEQ ID NO: 2 - protein, 163 aa, Moringa oleifera
MAKITLLLATFGLLLLLTNASIYRTTVELDEEADENQQQRCRQQFQTHQRLRACQRFIRRRTQGGGPLDEVEDEVDEIEEVVEPDQGPGRQPAFQRCCQQLRNISPPCRCPSLRQAVQLTHQQQGQVGPQQVRQMYRVASNIPSMCNLQPMSCLFRQQQSSWL

SEQ ID NO: 3 - protein, 163 aa, Moringa oleifera
MAKLTLLLATFALLVLLANASIYRTTVELDEEPDDNQQQRCRHQFQSQQRLRACQRVIRRWSQGGGPMEDVEDEIGETDEIEEVVEPDQARRPPTLQRCCRQLRNVSPFCRCPSLRQAVQSAQQQQGQVGPQQVGHMYRVASRIPAICNPQPMRCPFRQQQGS

SEQ ID NO: 4 - protein, 163 aa, Moringa oleifera; identical to SEQ ID NO: 2

SEQ ID NO: 5 - protein, 160 aa, Moringa oleifera
MAKFTLLLAIFALFLILANANVYRTTVELDEEPDDNQQGQQQQQCRQQFLTHQRLRACQRFIRRQTQGGGALEDVEDDVEEIEEVVEPDQARRPAIQRCCQQLRNIQPRCRCPSLRQAVQLAHQQQGQVGPQQVRQMYRLASNIPAICNLRPMSCPFGQQ

SEQ ID NO: 6 - protein, 163 aa, Moringa oleifera
MAKLTLLLATLALLVLLANASIYRTTVELDEEPDDNQQQRCRHQFQTQQRLRACQRVIRRWSQGGGPMEDVEDEIDETDEIEEVVEPDQARRPPTLQRCCRQLRNVSPFCRCPSLRQAVQSAQQQQGQVGPQQVGHMYRVASRIPAICNLQPMRCPFRQQQSS